(19) United States
(12) Patent Application Publication
(10) Pub. No.: US 2015/0367119 A1
(43) Pub. Date: Dec. 24, 2015

(54) TAMPER RESISTANT/EVIDENT INDICATOR FOR SELECTIVE ENGAGEMENT IN SECURING A CLAVE/IV NEEDLE ASSEMBLY OR INDEPENDENTLY TO RESTRICT AND INDICATE UNAUTHORIZED ACCESS AND USE

(71) Applicant: Randall Stillson, Whitefish, MT (US)

(72) Inventor: Randall Stillson, Whitefish, MT (US)

(21) Appl. No.: 14/310,232

(22) Filed: Jun. 20, 2014

(51) Int. Cl. A61M 39/10

(52) U.S. Cl. CPC A61M 39/1011

(57) ABSTRACT

A tamper resistant/evident indicator for selective engagement in securing a claven/IV needle assembly or independently to restrict and indicate unauthorized access and use. The indicator has a pair of hinged cover portions each with a matching contoured recessed interior for receiving and holding the claven/IV needle when positioned therewithin. Interlocking opposing latching frangible tabs extend from one cover portion and are receivably secured into the co-aligned receiving receptacles on the corresponding remaining cover portion. The tabs will confirm opening of the hinge cover after locking.

This application claims the benefit of U.S. Provisional Application No. 61/843,526, filed Jul. 8, 2013.

### BACKGROUND OF THE INVENTION

1. Technical Field

This invention relates to tamper resistant and therefore evident covers for needle equipment, specifically claven and IV needle assemblies.

2. Description of Prior Art

Prior art devices of this type have relied on a variety of different closure strategies; see for example U.S. Pat. Nos. 4,286,640, 5,513,695, 7,678,101 and 8,091,727.

In U.S. Pat. No. 4,286,640 a hinged tamper proof cover can be seen having a pair of hemispherical body members hinged together along adjacent sidewall edges.

U.S. Pat. No. 5,513,695 illustrates a tamper evident sleeve for IV needles that have a pair of elongated hinge covers with a hinge which is removable to access a needle by user engagement pull tab extending therefrom.

U.S. Pat. No. 7,678,101 is directed to a locking catheter connector and connector system.

Finally, in U.S. Pat. No. 8,091,727 a snap over clam shell protective port cap is claimed having two halves hinged together with a puncture resistant lid portion so as to cover when applied onto the end of an injection port.

### SUMMARY OF THE INVENTION

A tamper indicating and resistant claven/and IV needle cover for receiving and securing access thereto by preventing unauthorized access and use without direct evidence of such use. A hinged two-part cover assembly has corresponding recessed contoured areas therewithin to receive and hold the claven or IV needle independently or in assembly for complete locking access or in an alternate form of the invention when interengaged on associated medical equipment. Oppositely disposed frangible tabs extend from the cover portion for securing the cover portions together and must be broken to release same for access.

### DESCRIPTION OF THE DRAWINGS

FIG. 1 is an enlarged side elevational view thereof.

FIG. 2 is a top plan view thereof.

FIG. 3 is a perspective view showing the cover in use position for closing.

FIG. 4 is a perspective view in a closed position.

FIG. 5 is an end view on lines 5-5 of FIG. 1.

FIG. 6 is an end view on lines 6-6 of FIG. 1.

FIG. 7 is a perspective view of an alternate form of the invention.

### DETAILED DESCRIPTION OF PREFERRED EMBODIMENT

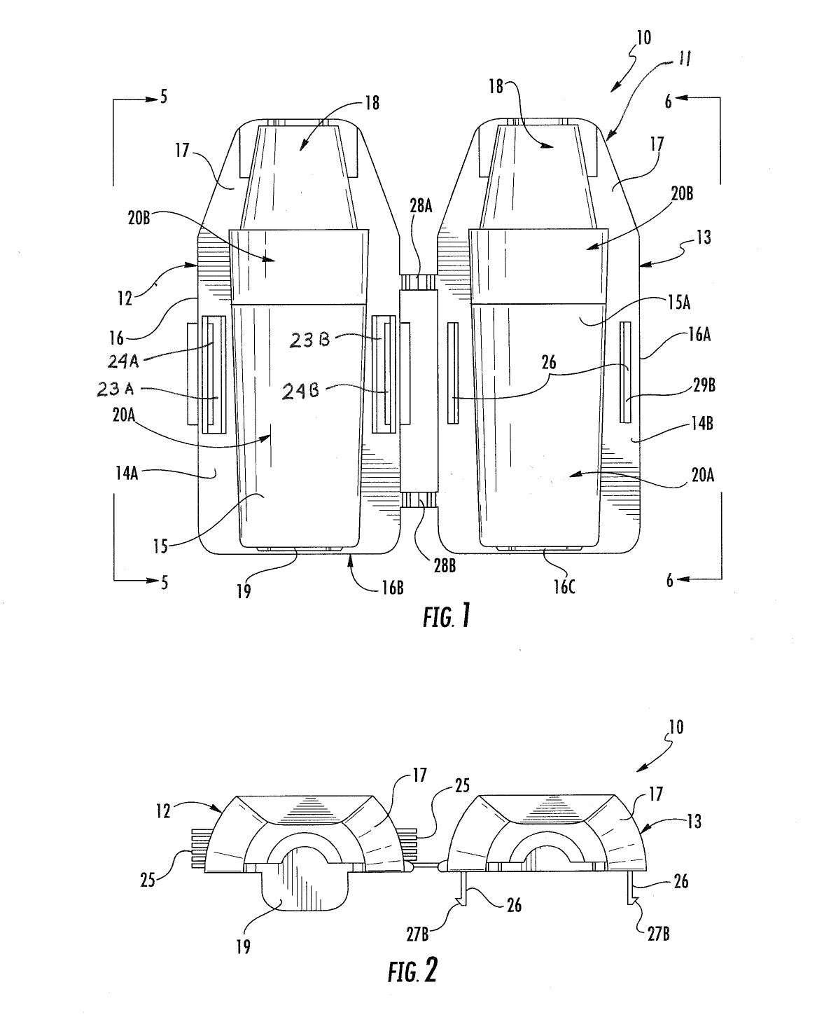

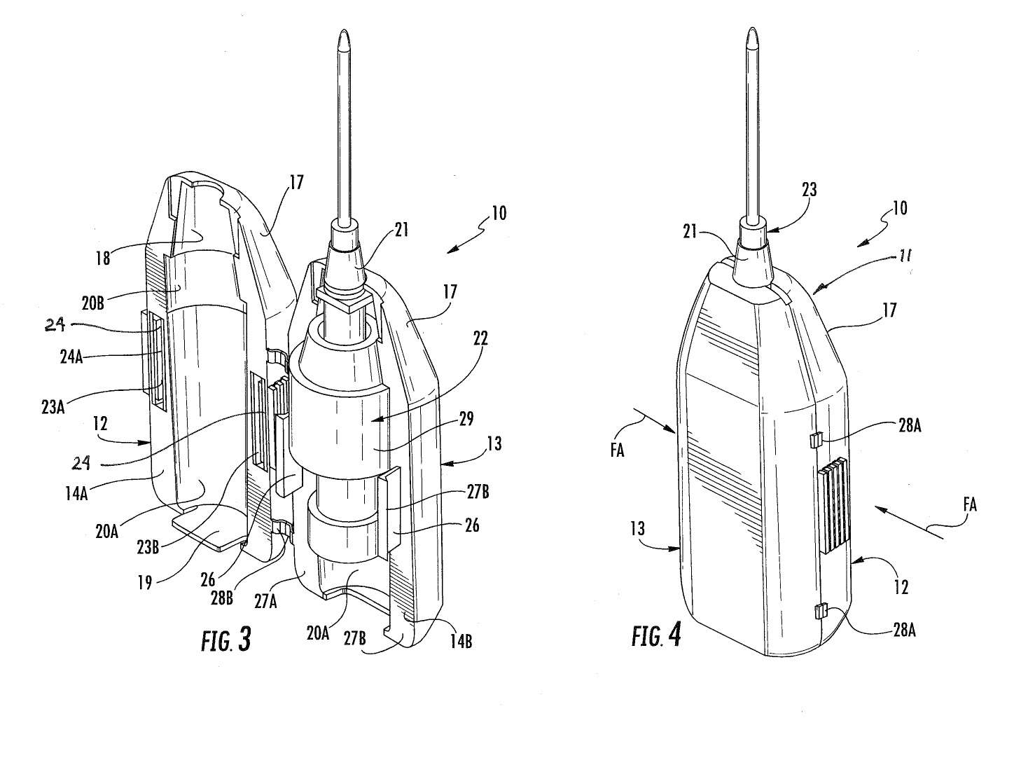

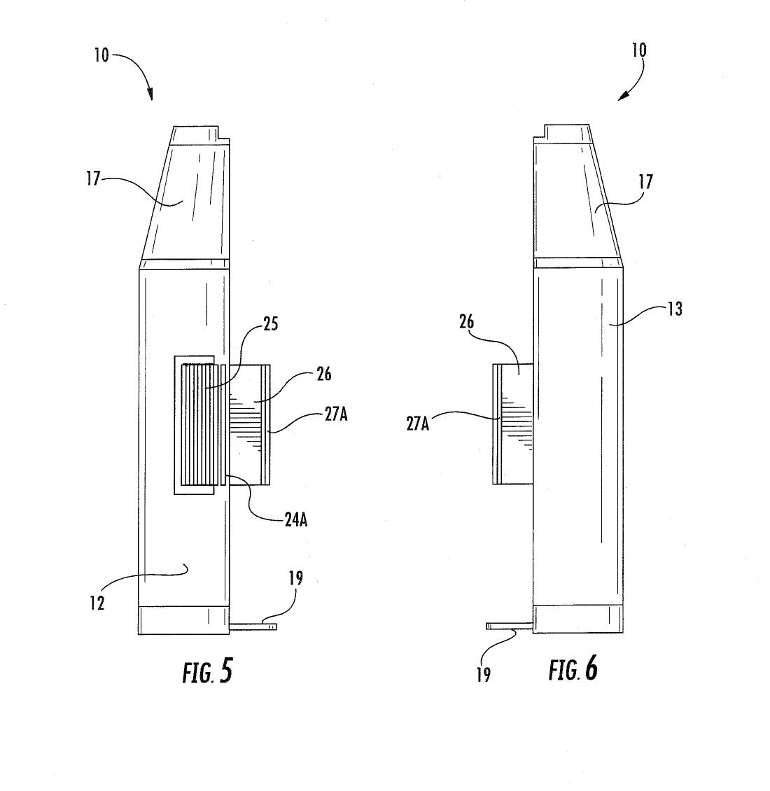

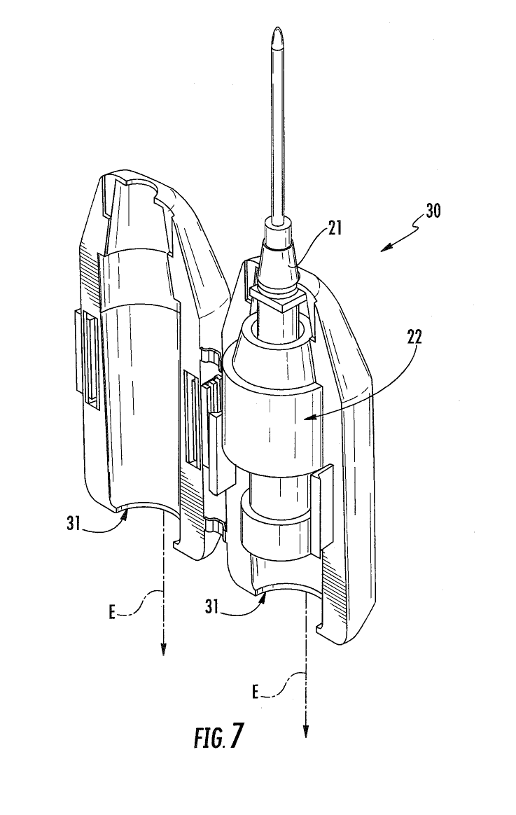

Referring to FIGS. 1 AND 2 of the drawings, a claven/IV needle locking device cover 10 of the invention can be seen having an interengaging sleeve 11 with a locking portion 12 and a clasp portion 13. The locking portion 12 and clasp portion 13 both have an aligned semi-cylindrical body members 14A and 14B with elongated half arcuate recessed areas 15 and 15A formed therein. Correspondingly, their respective exterior surfaces 16 and 16A have tapered end portions 17 defining a needle end access opening when closed at 18 as will be described in greater detail hereinafter. A closure end tab 19 extends outwardly from the oppositely disposed end 16B of the locking portion 12 best seen in FIGS. 2 and 3 of the drawings for registration within the clasp portion 13's aligned end area 16C, best seen in FIGS. 1, 2 and 3 of the drawings.

The interior surface of the recessed areas 15 and 15A have two adjacent areas of reduced arcuate interior diameter at 20A and 20B extending inwardly from the access opening portion therebetween at 18.

The adjoining needle end access opening portion 18 is tapered to provide clearance for the IV needle portion 21 of an illustrated claven IV needle assembly 22, shown positioned in FIG. 3 of the drawings prior to closing.

The locking portion 12 has oppositely disposed ear engagement tabs 24A and 24B formed in its respective side surfaces thereof and defined by longitudinal slots 23A and 23B. The respective ear engagement tabs 23A and 23B have ribbed surfaces 25 with respective interior clasps retainment registration longitudinally extending edges 25A and 25B therewithin.

Referring now to the clasp portion 13 of the cover locking device 10, a pair of clasp arms 26 extend from their respective space parallel free edge surfaces 27A and 27B of the hereinbefore described semi-cylindrical body member 14B with space parallel extending barbs 27A and 27B thereon. The respective clasp arms 26 are aligned for registration within and retainment with the respective hereinbefore slots 23A and 23B and edges 25A and 25B upon closure.

The latch portion 12 and clasp portion 13 are interconnected by a pair of longitudinally spaced live hinge elements 28A and 28B extend from correspondingly aligned adjacent free edges thereof as seen best in FIG. 1 of the drawings.

It will be seen that during use, the claven needle assembly 22 in this illustration is initially placed within the clasp portion 13 as seen in FIG. 3 of the drawings with the IV needle portion 21 extending out through the defined access opening portion 18. The latch portion 12 is then hinged over and engaged via the respective registration of the hereinbefore described clasp arms 26 within the locking slots as seen in FIG. 4 of the drawings. Once secured within the locking cover device 10 of the invention, unauthorized access to the claven/IV needle assembly 22 is not possible without indicating such access has occurred.

It will also be evident that the initial placement of the claven/IV needle assembly 22 can occur in the lock portion 12 depending on user preference and venue requirement.

Once so engaged as illustrated in FIG. 4 enclosed, the locking device cover 10 of the invention will prevent access to a claven portion 29 as well as restrict removal of the IV needle portion 21 therefrom.

To open the engaged locking device cover 10 of the invention, the respective ribbed surfaces 25 of the respective ear engagement tabs 24A and 24B are grasped by the user (not shown) but indicated by force arrows FA and compressed, breaking the clasp engaged arms 26 which are frangible, thus releasing the clasp portion 13 from the closed engagement with its corresponding lock portion 12 opening same and allowing removal and access to the claven IV needle assembly 22 as seen in FIG. 3 of the drawings.

It will be evident that if required either the IV needle portion 21 or the claven portion 29 can be independently secured within the locking device cover 10 of the invention, given its interior configuration as illustrated and described hereinabove.

Referring now to FIG. 7 of the drawings, and alternate form of the invention can be seen at 30 in the form of a modified fitting configuration.

The alternate locking fitting 30 is identical to the primary form of the invention illustrated above except for the removal of the closure end tab 19 which provides an oppositely disposed open access end at 31 but the claven IV needle assembly 22 illustrated in this example can be interconnected to extend elements indicated by arrow E as required yet provide tamper-evident security as set forth in the primary form of the invention described.

It will thus be seen that a new and novel integrated claven/IV needle locking cover device has been illustrated and described and it will be apparent to those skilled in the art that various changes and modifications may be made thereto without departing from the spirit of the invention.

Therefore I claim:

1. A tamper indicating and resistant cover for use with clave and IV needle assemblies comprising,
a first cover locking portion and a second cover clasp portion, each having a hollow semi-cylindrical shape and contoured interior surface hinged together,
locking slots in said cover locking portion,
ear engagement tabs within said locking slots,
clasp arms extending from said cover clasp portion in selective registrational alignment with said ear engagement tabs,
a needle end access opening formed between said cover portions when in closed position a closure end tab extending from said cover locking portion in oppositely disposed relation to said end access opening, said closure end tab registerable within said cover clasp portion when in closed position.

2. The tamper indicating and resistant cover set forth in claim 1 wherein said first cover locking portion and said second cover clasp portion have parallel spaced live hinge elements extending therebetween.

3. The tamper indicating and resistant cover set forth in claim 1 wherein said respective cover portions have adjacent areas of reduced arcuate interior diameter inwardly of said access end opening.

4. The tamper indicating and resistant cover set forth in claim 1 wherein said ear engagement tabs have textured exterior surface and interior extending longitudinal edges for locking engagement with said clasp arms when so engaged in closed position.

5. The tamper indicating and resistant cover set forth in claim 1 wherein said clasp arms have parallel extending barbs there across.

6. (canceled)

7. (canceled)

8. (canceled)

9. (canceled)

10. (canceled)

11. The tamper indicating and resistant cover set forth in claim 1 wherein said closure end tab registerable within said cover clasp portion in closed position, is arranged to block access to said cover interior surface therefrom.
